(19) United States
(12) Patent Application Publication
(10) Pub. No.: US 2016/0256123 A1
(43) Pub. Date: Sep. 8, 2016

(54) METHOD AND APPARATUS FOR STATIC 3-D IMAGING OF HUMAN FACE WITH CBCT

(71) Applicant: Carestream Health, Inc., Rochester, NY (US)

(72) Inventors: Yanbin Lu, Shanghai (CN); Qinran Chen, Shanghai (CN); Wei Wang, Shanghai (CN)

(21) Appl. No.: 15/047,676

(22) Filed: Feb. 19, 2016

Related U.S. Application Data
(60) Provisional application No. 62/129,088, filed on Mar. 6, 2015.

(51) Int. Cl. A61B 6/00; A61B 6/14; A61B 6/03

(52) U.S. Cl. CPC A61B 6/4417; A61B 6/032; A61B 6/4085; A61B 6/501; A61B 6/14; A61B 6/461

(57) ABSTRACT

An apparatus for imaging the head of a patient has a transport apparatus that moves an x-ray source and detector in at least partial orbit about a head supporting position for acquiring 2-D radiographic projection images of the head. A light source coupled to the transport apparatus projects patterned light over at least a portion of the orbit. A monochrome camera coupled to the transport apparatus records, at angles of the orbit, monochrome reflectance images of the projected patterned light. A color camera coupled to the transport apparatus acquires, at each of one or more angles of the orbit, a color reflectance image of the head. A control logic processor energizes at least the x-ray source, the detector, the transport apparatus, the light source, and the cameras to acquire and process both radiographic and reflectance image data obtained during the at least partial orbit about the head supporting position.

### CROSS REFERENCE TO RELATED APPLICATIONS

This application claims priority to U.S. Patent Application Ser. No. 62/129,088, filed Mar. 6, 2015, in the name of Lu et al., and entitled METHOD AND APPARATUS FOR STATIC 3-D IMAGING OF HUMAN FACE WITH CBCT.

### TECHNICAL FIELD

The disclosure relates generally to the field of volume imaging and more particularly to methods and apparatus for combining volume images that have been reconstructed from radiographic projection images of the human head of a patient with contour and texture image content obtained from the outer surface of the patient's face.

### BACKGROUND

Radiological imaging is recognized to have significant value for the dental practitioner, helping to identify various problems and to validate other measurements and observations related to the patient's teeth and supporting structures. Among x-ray systems with particular promise for improving dental care is the extra-oral imaging apparatus that is capable of obtaining one or more radiographic images in series and, where multiple images of the patient are acquired at different angles, combining these images to obtain a 3-D reconstruction showing the dentition of the jaw and other facial features for a patient. Various types of imaging apparatus have been proposed for providing volume image content of this type. In these types of systems, a radiation source and an imaging detector, maintained at a fixed distance from each other, synchronously revolve about the patient over a range of angles, taking a series of images by directing and detecting radiation that is directed through the patient at different angles of revolution. For example, a volume image that shows the shape and dimensions of the head and jaws structure can be obtained using computed tomography (CT), such as cone-beam computed tomography (CBCT), or other volume imaging method, including magnetic resonance imaging (MRI) or magnetic resonance tomography (MRT).

While 3-D radiographic imaging techniques can be used to generate volume images that accurately show internal structure and features, however, there is a need to relate the radiographic volume image data with the patient's facial structure and external appearance. The volume image that is generated from a CT, CBCT, or other volume imaging apparatus has no color or perceptible textural content and would not, by itself, be of much value for showing simulated results of a procedure to a patient or other non-practitioner, for example. Communication between the practitioner and patient can be constrained without some way of showing how a proposed procedure will affect the patient's face.

Generating a volume image that also provides a suitable visualization of the human face for planning and implementing corrective procedures relating to teeth, jaws, and related dentition can require multiple types of imaging. Internal structure is obtained using radiographic techniques with CT, CBCT, and similar imaging apparatus. In addition, in order to provide useful visualization that incorporates the outer, textural surface of the human face, two other types of imaging are used. A camera is used to obtain reflectance or “white light” images. The color and texture information from the camera images can then be correlated with volume image information in order to provide an accurate rendition usable by the practitioner. To provide surface contour information that allows proper correlation of the reflectance image content with the radiographic image content, additional contour image data can also be obtained, such as by using a scanning technique or other method for mapping the patient's facial contour.

Solutions that have been proposed for addressing this problem include methods that provide at least some level of color and texture information that can be correlated with volume image data from CBCT or other scanned image sources. These conventional solutions include so-called range-scanning methods.

Reference is made to U.S. Patent Application Publication No. 2012/0300895 entitled “DENTAL IMAGING APPARATUS” by Koivisto et al. that combines texture information from reflectance images along with surface contour data from a laser scan.

Reference is made to U.S. Patent Application Publication No. 2013/0163718 entitled “DENTAL X-RAY DEVICE WITH IMAGING UNIT FOR SURFACE DETECTION AND METHOD FOR GENERATING A RADIOGRAPH OF A PATIENT” by Lindenberg et al. that describes using a masking edge for scanning to obtain contour and color texture information for combination with x-ray data.

The '0895 Koivisto et al. and '3718 Lindberg et al. patent applications describe systems that can merge volume image data from CBCT or other scanned image sources with 3-D surface data that is obtained from 3-D range-scanning devices. The range scanning devices can provide some amount of contour data as well as color texture information. However, the solutions that are described in these references can be relatively complex and costly. Requirements for additional hardware or other specialized camera equipment with this type of approach add cost and complexity that may not be acceptable to practitioners.

A dental imaging system from Dolphin Imaging Software (Chatsworth, Calif.) provides features such as a 2-D facial wrap for forming a texture map on the facial surface of a 3-D image from a CBCT, CT or MRI scan. The software user, working with a mouse, touch screen, or other pointing device, must accurately align and re-position the 2-D content with respect to 3-D content that appears on the display screen. With this type of system, imprecise registration of 2-D data that provides information on image texture to the 3-D volume data can significantly compromise the appearance of the combined data.

Reference is made to a paper by Iwakiri, Yorioka, and Kaneko entitled “Fast Texture Mapping of Photographs on a 3D Facial Model” in Image and Vision Computing NZ, November 2003, pp. 390-395.

In conventional approaches such as those just described, some limited degree of success has been obtained for acquiring, correlating, and displaying the different types of image data that are needed for accurate representation of both internal structures and external facial appearance. However, at least for reasons of cost, usability, and performance, there is considered to be room for improvement.

### SUMMARY

An aspect of this application is to advance the art of medical digital radiography, particularly for dental applications.

Another aspect of this application is to address, in whole or in part, at least the foregoing and other deficiencies in the related art.

It is another aspect of this application to provide, in whole or in part, at least the advantages described herein.

It is an object of the present disclosure to advance the art of volume imaging and visualization used in medical and dental applications. Embodiments of the present disclosure address the particular need for improved visualization of the head of the patient, wherein internal structures obtained using CBCT and other radiographic volume imaging methods can be correlated to reflective images of the head and face surface. By combining volume image data with reflective image data, embodiments of the present disclosure can help the medical or dental practitioner and patient to visualize the effect of a procedure on patient appearance.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

According to one aspect of the disclosure, there is provided an apparatus for imaging the head of a patient, comprising:
a transport apparatus that moves an x-ray source and an x-ray detector in at least partial orbit about a head supporting position for the patient for acquiring, at each of a plurality of angles about the supporting position, a 2-D radiographic projection image of the patient's head;
a light source coupled to the transport apparatus and energizable to project a patterned light toward the head supporting position over at least a portion of the orbit;
a monochrome camera coupled to the transport apparatus and disposed to record, at each of one or more angles of the orbit, a monochrome reflectance image of the projected patterned light against the patient's head;
a color camera coupled to the transport apparatus and disposed to acquire, at each of one or more angles of the orbit, a color reflectance image of the patient's head at the head supporting position;
and
a control logic processor that energizes at least the x-ray source, the detector, the transport apparatus, the light source, and the monochrome and color cameras to acquire and process both radiographic and reflectance image data obtained during the at least partial orbit about the head supporting position.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other.

FIG. 1 is a schematic diagram that shows an imaging apparatus for CBCT imaging of a patient.

FIGS. 2A, 2B, 2C, and 2D show top view schematics of transport apparatus rotating about the patient's head and head supporting position.

FIG. 3A is a top view schematic showing components of the imaging apparatus for obtaining radiographic and reflectance image content.

FIG. 3B is a top view schematic showing components of the imaging apparatus according to an alternate embodiment of the present disclosure.

FIG. 4A is a schematic diagram that shows components for surface contour imaging using a light source.

FIG. 4B is a schematic diagram that shows components for surface contour imaging using a laser.

FIG. 4C is a schematic diagram that shows components for color reflectance imaging.

FIG. 5 is a perspective view that shows imaging component positioning according to an embodiment of the present disclosure.

FIG. 6A shows how contour imaging is executed using a pattern of projected lines.

FIG. 6B shows a collection of lines used to form various types of patterns for surface contour imaging.

FIG. 7 is a logic flow diagram that shows a sequence for forming a composite image.

FIG. 8 is a logic flow diagram that shows an image capture sequence that can repeat at discrete angles of transport apparatus rotation.

FIG. 9 shows an exemplary display of a composite image that would include both radiographic volume image content and contour and color reflectance image content.

### DETAILED DESCRIPTION OF THE EMBODIMENTS

The following is a detailed description of the preferred embodiments, reference being made to the drawings in which the same reference numerals identify the same elements of structure in each of the several figures.

Where they are used, the terms “first”, “second”, and so on, do not necessarily denote any ordinal or priority relation, but may be used for more clearly distinguishing one element or time interval from another. As used herein, the term “energizable” relates to a device or set of components that perform an indicated function upon receiving power and, optionally, upon receiving an enabling signal. The opposite state of “energizable” is “disabled”.

The term “actuable” has its conventional meaning, relating to a device or component that is capable of effecting an action in response to a stimulus, such as in response to an electrical signal, for example.

The term “modality” is a term of art that refers to types of imaging. Modalities for an imaging system may be conventional x-ray, fluoroscopy, tomosynthesis, tomography, ultrasound, MMR, contour imaging, color reflectance imaging, or other types of imaging. The term “subject” refers to the patient who is being imaged and, in optical terms, can be considered equivalent to the “object” of the corresponding imaging system.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components or fields.

It will be understood that when an element is referred to as being “connected,” or “coupled,” to another element, it can be directly connected or coupled to the other element or intervening elements or magnetic fields may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The term “exemplary” indicates that the description is used as an example, rather than implying that it is an ideal.

The term “in signal communication” as used in the application means that two or more devices and/or components are capable of communicating with each other via signals that travel over some type of signal path. Signal communication may be wired or wireless. The signals may be communication, power, data, or energy signals which may communicate information, power, and/or energy from a first device and/or component to a second device and/or component along a signal path between the first device and/or component and second device and/or component. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the terms “pixel” and “voxel” may be used interchangeably to describe an individual digital image data element, that is, a single value representing a measured image signal intensity. Conventionally an individual digital image data element is referred to as a voxel for 3-dimensional or volume images and a pixel for 2-dimensional (2-D) images. Volume images, such as those from CT or CBCT apparatus, are formed by obtaining multiple 2-D images of pixels, taken at different relative angles, then combining the image data to form corresponding 3-D voxels. For the purposes of the description herein, the terms voxel and pixel can generally be considered equivalent, describing an image elemental datum that is capable of having a range of numerical values. Voxels and pixels have attributes of both spatial location and image data code value.

In the context of the present disclosure, the term “code value” refers to the value that is associated with each volume image data element or voxel in the reconstructed 3-D volume image. The code values for CT images are often, but not always, expressed in Hounsfield units (HU). “Static” imaging relates to images of a subject without consideration for movement. “Patterned light” is used to indicate light that has a predetermined spatial pattern, such that the light has one or more features such as one or more discernable parallel lines, curves, a grid or checkerboard pattern, or other features having areas of light separated by areas without illumination. In the context of the present disclosure, the phrases “patterned light” and “structured light” are considered to be equivalent, both used to identify the light that is projected onto the head of the patient in order to derive contour image data.

In the context of the present disclosure, the terms “interference filter” and “dichroic filter” are considered to be synonymous. In the context of the present disclosure, the terms “digital sensor” or “sensor panel” and “digital x-ray detector” or simply “digital detector” are considered to be equivalent. These describe the panel that obtains image data in a digital radiography system. The term “revolve” has its conventional meaning, to move in a curved path or orbit around a center point. In the context of the present disclosure, the terms “viewer”, “operator”, and “user” are considered to be equivalent and refer to the viewing practitioner, technician, or other person who views and manipulates an x-ray image or a volume image that is formed from a combination of multiple x-ray images, on a display monitor. A “viewer instruction” or “operator command” can be obtained from explicit commands entered by the viewer or may be implicitly obtained or derived based on some other user action, such as making a collimator setting, for example. With respect to entries on an operator interface, such as an interface using a display monitor and keyboard, for example, the terms “command” and “instruction” may be used interchangeably to refer to an operator entry.

In the context of the present disclosure, a single projected line of light is considered a “one dimensional” pattern, since the line has an almost negligible width, such as when projected from a line laser, and has a length that is its predominant dimension. Two or more of such lines projected side by side, either simultaneously or in a scanned arrangement, provide a two-dimensional pattern.

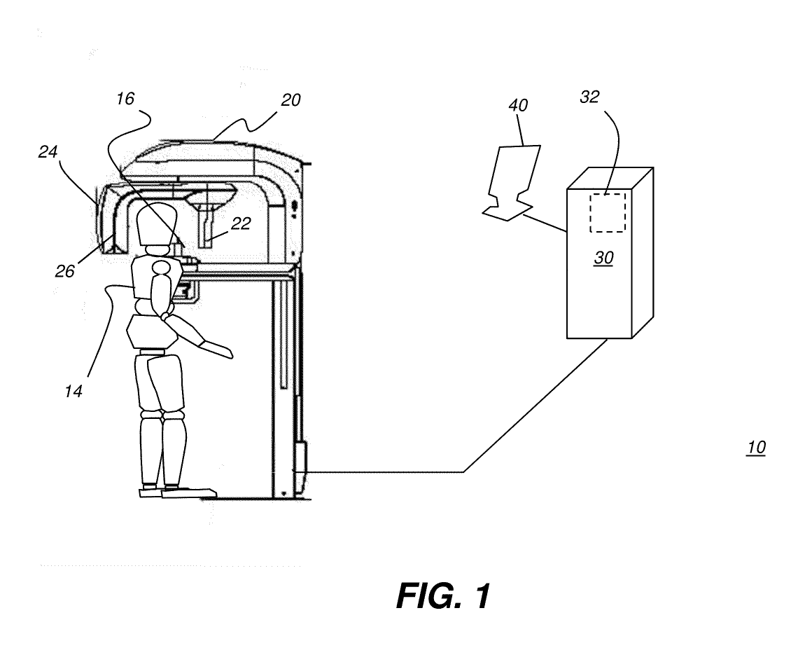

The schematic diagram of FIG. 1 shows an imaging apparatus 10 for acquiring, processing, and displaying a CBCT image of a patient 14. A transport apparatus 20 rotates a detector 22 and a generator apparatus 24 having an x-ray source 26 at least partially about a head supporting position 16 in order to acquire multiple 2-D projection images used for 3-D volume image reconstruction.

A control logic processor 30 energizes x-ray source 26, detector 22, transport apparatus 20, and other imaging apparatus for reflectance image illumination and acquisition, as described in more detail subsequently, in order to obtain the image content needed for static 3-D imaging of the patient's face. To standardize patient 14 position at a suitable location for imaging, stabilize head position, and to provide a reference for orbiting the detector 22 and source 26 about the patient's head with suitable geometry for imaging, head supporting position 16 can include features such as a temple support and other supporting structures. Head supporting position 16 is a location at which the patient's head is located; however, there may or may not be features provided at head supporting position 16 for constraining movement of the head during imaging. Control logic processor 30 is in signal communication with a display 40 for entry of operator instructions and display of image results.

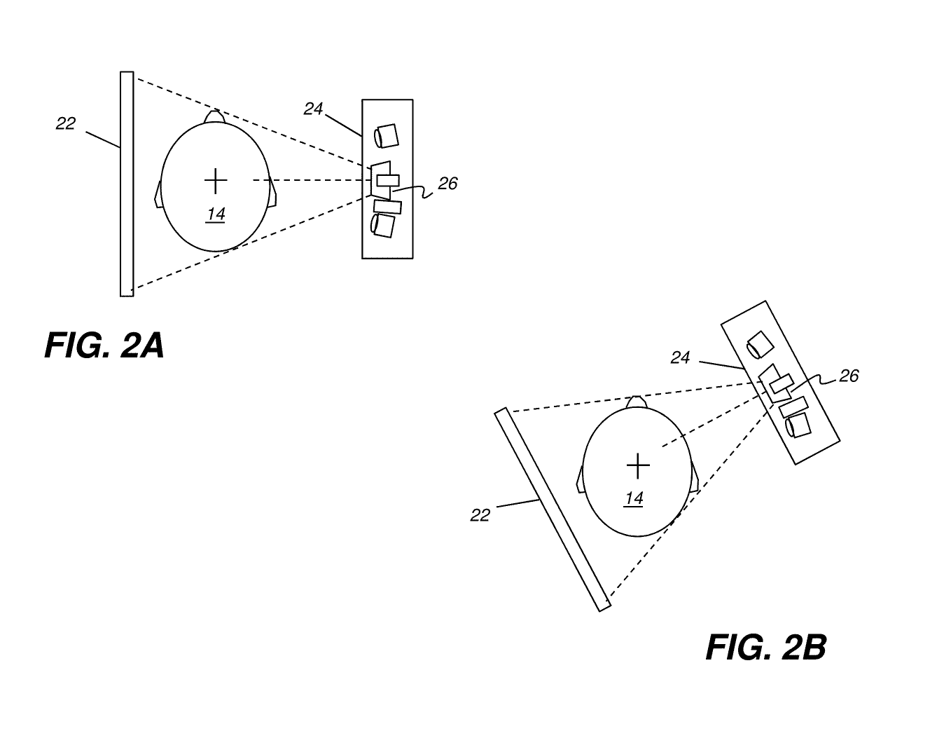

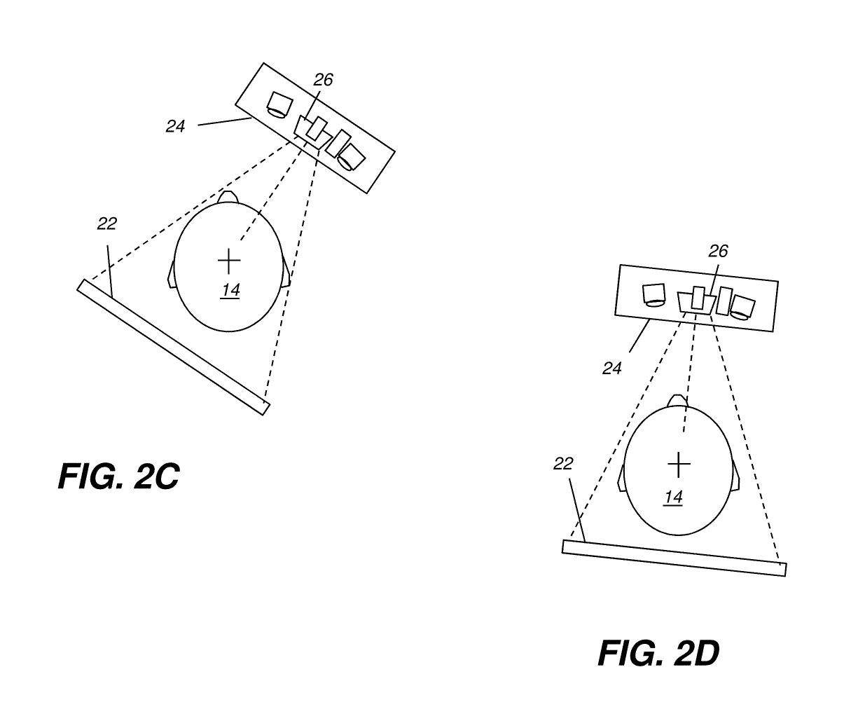

FIGS. 2A, 2B, 2C, and 2D show, for a few exemplary angles in top view schematic form, the action of transport apparatus 20 in orbiting generator apparatus 24 and detector 22 about the head of the patient 14 that is in head supporting position 16. The relative positions for generator apparatus 24 and detector 22 at four different representative angles are shown in FIGS. 2A-2D. At periodic angular increments during this rotation, x-ray source 26 is energized and detector 22 acquires the corresponding image content for the exposure at that angle. In the arrangement shown in FIG. 1, control logic processor 30 obtains the image data for each exposure, storing the 2-D projection image data for each exposure in a memory 32 for subsequent processing in order to generate the volume image content for display.

It should be noted that the orbit of generator apparatus 24 and detector 22 about the head of patient 14 is typically a circular orbit, but may alternately have a different shape. For example, the orbit can be in the shape of a polygon or ellipse or some other shape. Different portions of the orbit can be used for the different types of image acquisition that are performed. In practice, radiographic imaging for CBCT reconstruction is performed over a range of angles. However, a small portion of the orbit can be used in some cases, such as imaging from a single angle for acquiring some types of reflectance images, to a substantial portion of the orbit, such as acquiring reflectance images at numerous incremental angles about the head, over a portion of the orbit that extends from one side of the head to the other.

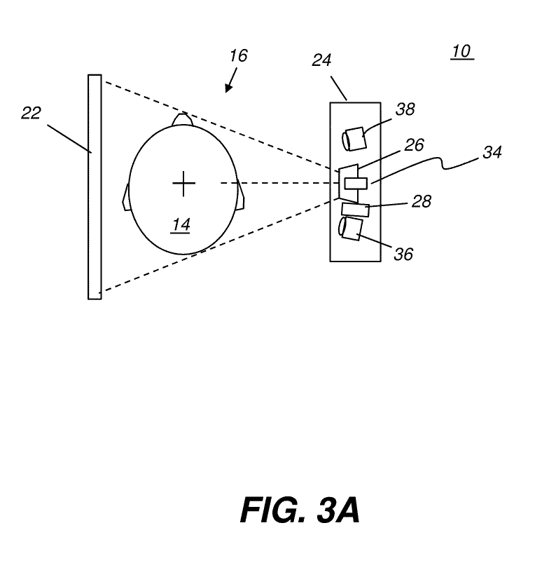

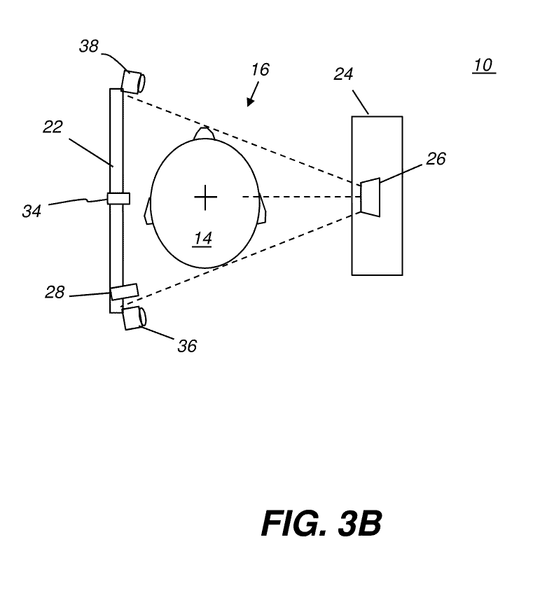

The top view schematic diagrams of FIGS. 3A and 3B show some of the components of imaging apparatus 10 in more detail. Generator apparatus 24 houses x-ray source 26, as noted previously, and can include additional components for obtaining reflectance image content for facial surface contour, color, and texture from patient 14 at head supporting position 16 as shown in FIG. 3A. Alternately, one or more of these additional imaging components can be provided adjacent to detector 22 as shown in FIG. 3B. An optional white light source 28, such as an LED or set of LEDs, provides polychromatic or “white” light for obtaining reflectance images at a color camera 36. These reflectance images provide texture and color content for mapping onto the surface contour image content that is generated from a light source 34 patterns that are acquired by a monochrome camera 38.

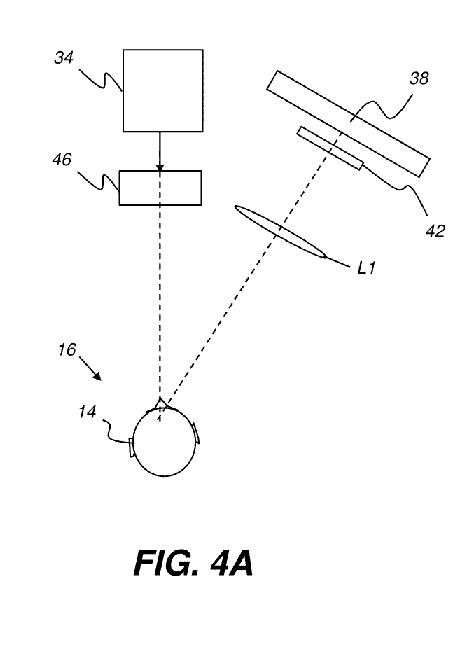

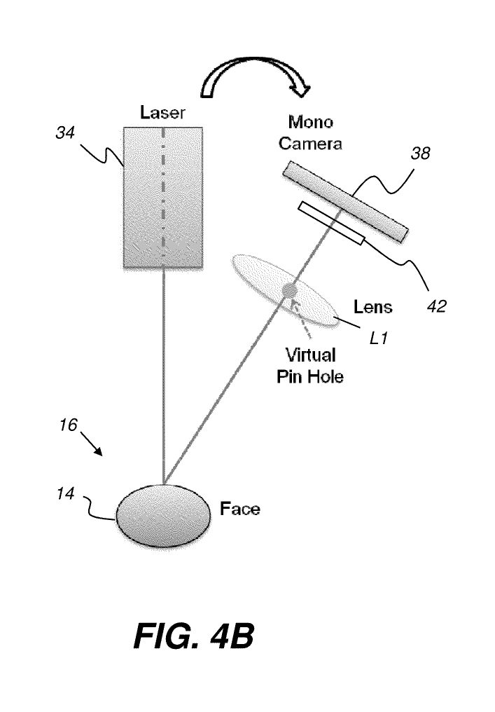

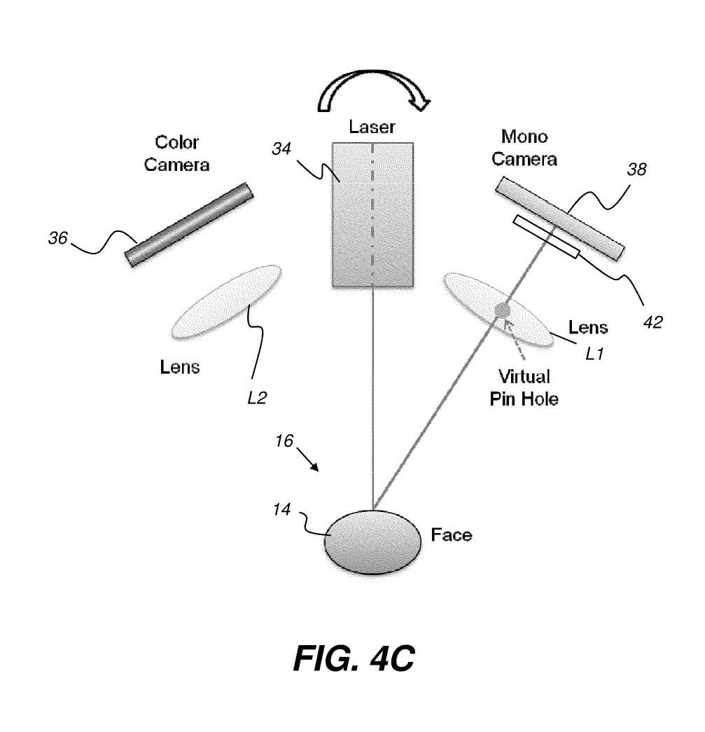

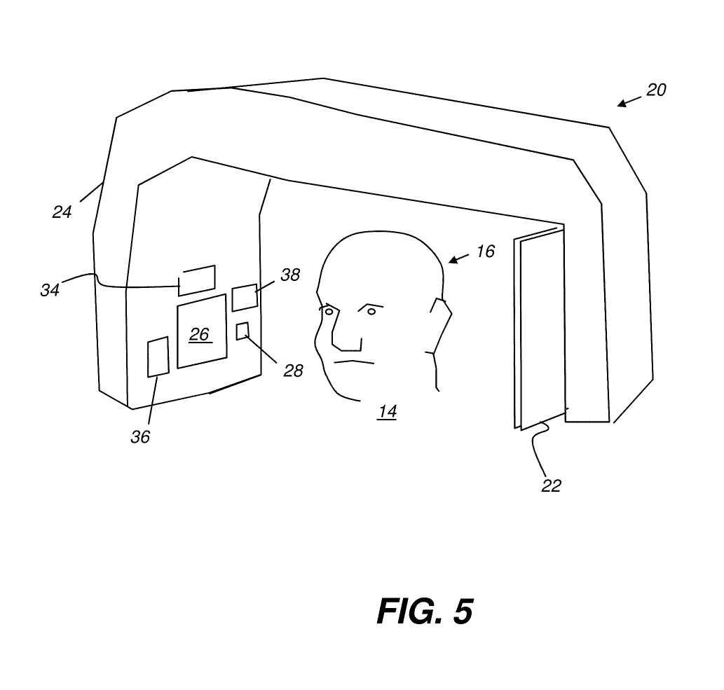

The schematic diagrams of FIGS. 4A-4C and perspective view of FIG. 5 show components used to obtain surface contour and color reflectance image content for the face of patient 14 at head supporting position 16 according to an embodiment of the present disclosure. A light source 34, which may be a solid-state light source such as a Light-Emitting Diode (LED) or laser source, directs light through an optional light conditioning element 46 that can provide a patterned light, also termed a structured light, that is then directed toward the head of patient 14 at head supporting position 16. Light conditioning element 46 can be a spatial light modulator, such as a Digital Light Processor (DLP) from Texas Instruments, Inc., Dallas Tex. or other micromirror array, or a liquid crystal device (LCD) array, for example. Alternately, light conditioning element 46 can be a grating or other device that forms a patterned or structured light when used in conjunction with light from light source 34. The structured light pattern that is generated can be a one-dimensional (1-D) pattern, such as a single line at a time or a 2-D multiline pattern, or other type of 2-D pattern, including a grid or checkerboard pattern, for example. Light conditioning element 46 can alternately be a scanner or other device that progressively forms a patterned light for projection onto a surface. A lens L1 directs light from the surface through an optional filter 42, such as a notch filter, and to monochrome camera 38. FIG. 4B shows an alternate embodiment that uses a laser as light source 34. Laser light source 34 directs a pattern of light, such as a line pattern, toward patient 14 in head supporting position 16. Camera 38 acquires an image of the projected line pattern through lens L1, which may be the camera 38 objective lens or other light-directing components. Lens L1 provides a virtual pinhole in the imaging optics path. The reflected laser light can be incident on an optional optical filter 42, such as a band pass or long wavelength pass (LWP) interference or dichroic filter, for example.

According to an embodiment of the present disclosure, light source 34 is a near infrared (NIR) laser of Class 1, with a nominal emission wavelength of 780 nm, well outside the visible spectrum. Light from this type of light source 34 can be projected onto the patient's face without awareness of the patient and without concern for energy levels that are considered to be perceptible or harmful at Class 1 emission levels. Infrared or near infrared light in the 700-900 nm region appears to be particularly suitable for surface contour imaging of the head and face, taking advantage of the resolution and accuracy advantages offered by the laser, with minimal energy requirements. It can be appreciated that other types of lasers and light sources, at suitable power levels and wavelengths, can alternately be used.

Light source 34 is shown coupled to generator apparatus 24 in the embodiments shown in FIGS. 3A and 5. However, it should be noted that light source 34 can be coupled to transport apparatus 20 in some other position for orbital motion about the head supporting position 16.

The schematic diagram of FIG. 4C shows the addition of a color camera 36 and optional polychromatic light source 28 to the imaging devices that are housed in generator apparatus 24. As shown in FIG. 4C, camera 36 has an associated lens L2 for color reflectance image acquisition.

The perspective view of FIG. 5 shows how the additional cameras 36, 38 and their associated light sources 26 and 34 are arranged as part of generator apparatus 24, according to an embodiment of the present disclosure. As noted previously, alternate arrangements of these optical components can be used. For example, a single camera can be used for acquiring both contour image content and color image data, as shown in FIG. 4A Contour image content can be obtained using a monochrome camera 38 or a suitably equipped color camera 36.

In surface contour imaging, according to an embodiment of the present disclosure, light source 34 projects one 1-D line of light at a time onto the patient or, at most, not more than a few lines of light at a time, at a particular angle, and acquires an image of the line as reflected from the surface of the patient's face or head. This process is repeated, so that a succession of lines is obtained for processing as transport apparatus 20 moves the light source 34 source to different angular positions. Other types of pattern can be projected, including irregularly shaped patterns or patterns having multiple lines. Light source 34 can be provided with an appropriate lens for forming a line, such as with a cylindrical lens or aspheric lens such as a Powell lens, for example. Additional optical components can be provided for shaping the laser output appropriately for contour imaging accuracy. The laser light can also be scanned across the face surface, such as using a rotating reflective scanner, for example. Scanning can be along the line or orthogonal to line direction.

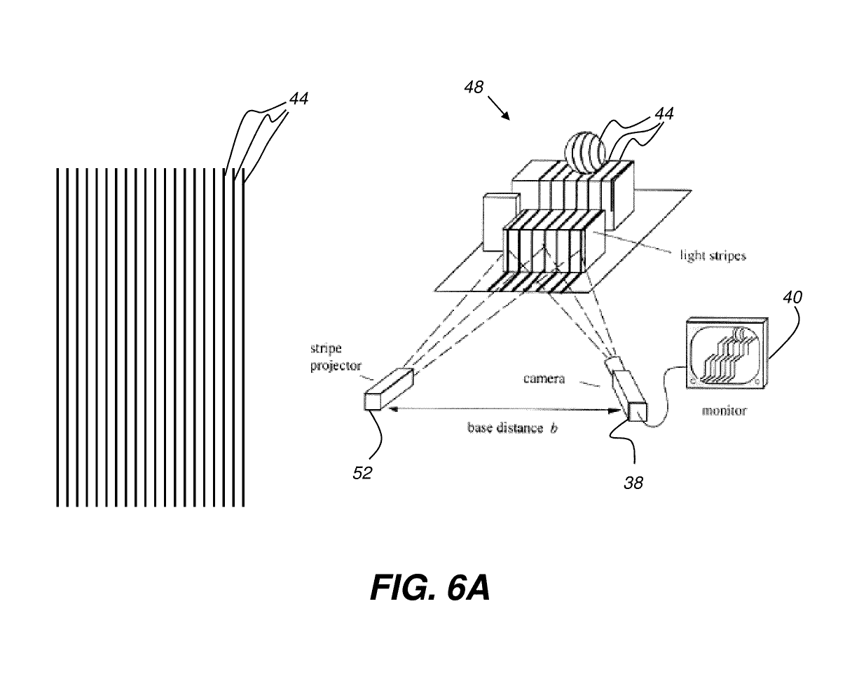

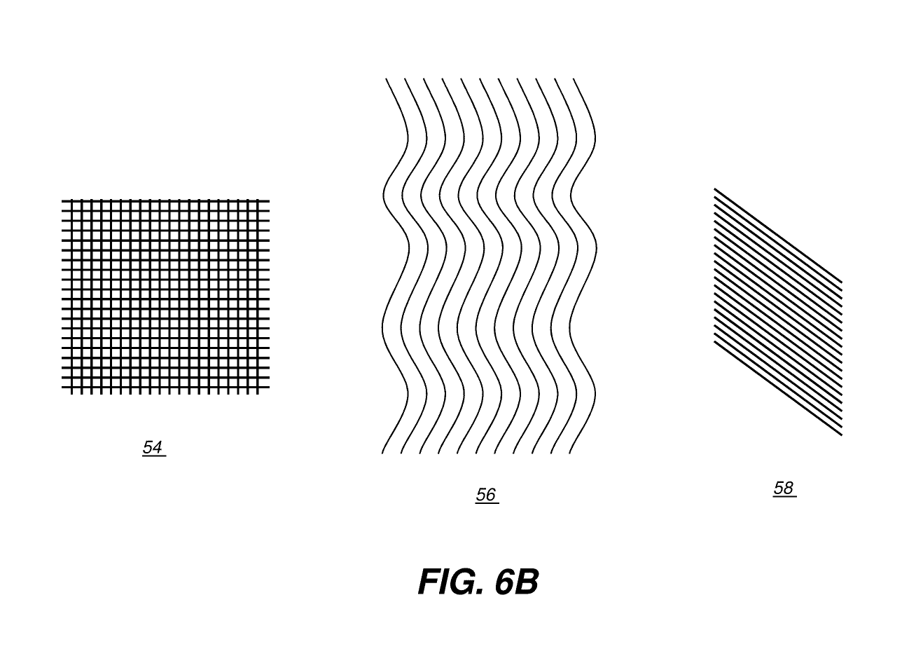

FIG. 6A shows, in simulated form, how surface contour imaging can be provided from a projector 52 using lines 44 individually projected from a laser source at different orbital angles toward a surface 48, represented by multiple geometric shapes. The combined line images, taken from different angles but registered to geometric coordinates of the imaging system, provide structured light pattern information. Triangulation principles are employed in order to interpret the projected light pattern and compute head and facial contour information from the detected line deviation. Lines 44 can be invisible to the patient and to the viewer, as well as to color camera 36 (FIG. 5).

The use of light outside the visible spectrum for forming lines 44 or other laser light pattern can be advantageous from a number of aspects. Lines 44 can be detected on a camera 38 that is sensitive to light at a particular wavelength, such as using one or more filters in the imaging light path.

FIG. 6B shows some of the other light patterns that can be projected onto the patient's face and used for surface contour imaging. Some of the 2-D light patterns that can be used include a grid 54 or checkerboard pattern, an arcuate pattern 56, and an oblique pattern 58, for example. Other possible patterns include patterns with scanned lines in different directions and patterns with lines of different thicknesses, different interline distances, and various types of encodings, for example. Sets of lines can be parallel or piece-wise parallel, such that adjacent segments of the projected line features extend in parallel directions. Light conditioning element 46 in FIG. 4A can be used to control the line pattern that is used, as described previously.

According to an alternate embodiment of the present invention, a single color camera 36 can be configured to provide the function described earlier for monochrome camera 38. Filtering can be provided, for example, to allow camera 36 to alternately capture color and texture content and capture patterned line content, or to capture both simultaneously, without perceptible impact on color quality.

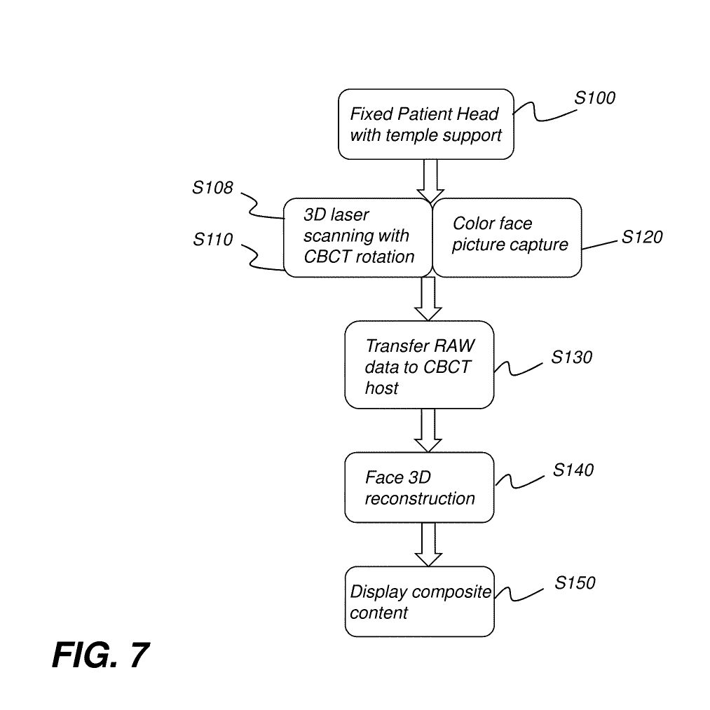

The logic flow diagram of FIG. 7 shows a sequence of processing tasks that can provide combined CBCT, contour image, and color image content using the apparatus and methods of the present disclosure. In a preparation step S100, the patient is positioned within head support position 16 (FIG. 1). In a CBCT acquisition step S108, the 2-D projection images needed for CBCT imaging are acquired at incremental exposure angles. A contour imaging step S110 obtains contour image content, as reflectance images, using light source 34 and monochrome camera 38. Steps S108 and S110 can be concurrently executed, so that the projection radiographic images for CBCT and the reflectance images for contour image content are acquired during the same rotation cycle of transport apparatus 20. A reflectance imaging step S120 obtains color images of the patient's head from various angles. Step S120 can similarly be executed simultaneously with either or both Steps S108 and S110.

According to an embodiment of the present disclosure, the 2-D projection images for volume reconstruction, monochrome reflectance images for contour imaging content, and color reflectance images for color and texture content are all acquired in one single rotation of transport apparatus 20. This allows the image content of each type to be in register using known coordinates of the imaging apparatus 10 and its transport assembly 20 angle, so that the reflective image content can readily be spatially correlated to the 3-D volume reconstruction. Continuing with FIG. 7, in a data transfer step S130, the acquired image data is transferred to control logic processor 30 (FIG. 1) for processing. Step S130 can be executed during image acquisition in steps S108, S110, and S120. A reconstruction step S140 is then executed on control logic processor 30, forming a volume image from the 2-D projection images acquired from step S108. Reconstruction step S140 can use some well known reconstruction algorithm for forming a volume image, such as filtered back projection, Feldkamp-Davis-Kreis (FDK) reconstruction, or algorithmic reconstruction techniques, for example. Reflectance image content from contour and color imaging steps S110 and S120 is then mapped to the volume image content in reconstruction step S140 to provide a composite image for display in a display step S150.

It should also be noted that, although there can be advantages to acquiring both reflectance and radiographic image content during the same scan about the patient's head, the different types of image acquisition can be separately performed and merged as a separate process.

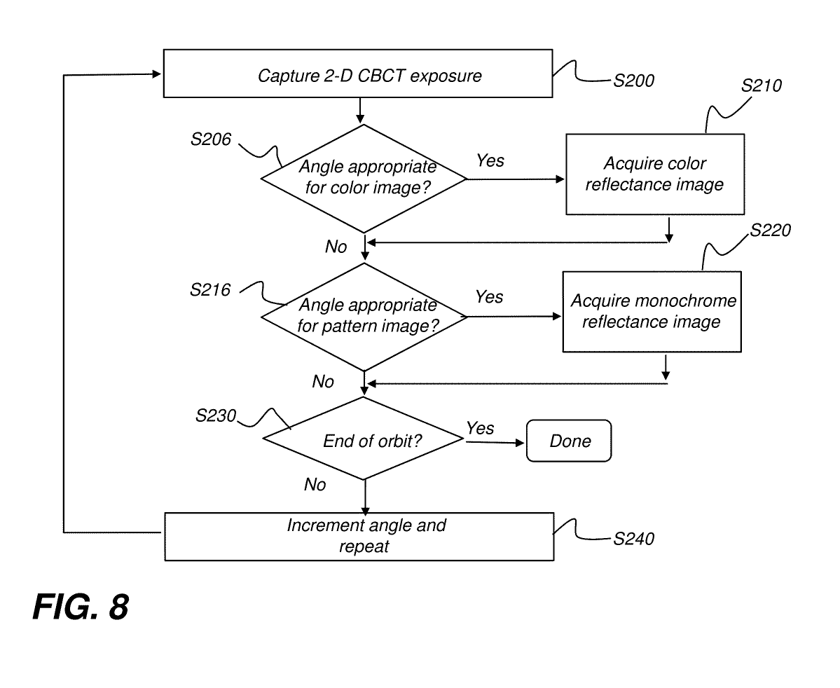

FIG. 8 is a logic flow diagram that shows, by way of example and not of limitation, an image capture sequence that allows image acquisition of CBCT projection images, color reflectance images, and structured light images in a single orbit of transport apparatus 20 about the patient (FIG. 1). The basic sequence shown in FIG. 8 is based on the assumption that reflectance images are needed at wide angular increments, in comparison to radiographic images used for CBCT reconstruction. The cycle in FIG. 8 repeats for angular positions in the orbit of transport apparatus 20. In a radiographic image capture step S200, the x-ray source 26 is energized to generate and direct radiation through patient 14 and to detector 22 for acquiring a single 2-D projection image of radiographic image data. An angular determination step S206 then determines whether or not color image capture is needed at the current angular position. At angular increments where color image capture is appropriate, a reflectance imaging step S210 executes. In reflectance imaging step S210, white light source 28 is energized and color camera 36 acquires a color reflectance image. If step S206 determines that a color reflectance image is not needed, or following step S210 if executed, the process continues to a second determination step S216. In second angular determination step S216, the system determines whether or not structured light image content is needed at the current angular position. At angular increments where structured light image capture is appropriate, a structured light imaging step S220 executes, in which light source 34 is energized and monochrome camera 38 acquires a structured light image for use in contour computation. Otherwise, the process continues to a decision step S230. At decision step S230, processing logic determines whether or not the full orbit has executed. If not, an angle increment step S240 executes, moving transport apparatus 20 to the next angle for a repeated imaging sequence. It can be appreciated that this sequence is one of many possible imaging sequences that can be executed by imaging apparatus 20 for acquiring the different types of image data that are needed in order to form the composite image of the present disclosure. For example, different types of images can be acquired at different angles and accurately registered to each other, since the different images are obtained on the same imaging system so that imaging geometry is well known. Image processing for each type of obtained image data can be executed as the image data is collected or once image data acquisition is complete.

It must be emphasized that the logic flow diagram of FIG. 8 shows one possible sequence for image acquisition; other sequences can also be used for obtaining the image data that is needed for relating surface data to the CBCT image content. For example, the color and contour image content can be obtained from a separate scan of the patient's head, either before or after the CBCT scan.

According to an alternate embodiment of the present disclosure, the contour image content is obtained from a single angular position. To accomplish this, a structured light image is obtained by projecting a 2-D pattern against the head of the patient. Depending on the angle of the source and camera and on the pattern that is projected, this method may provide sufficient contour image for a portion of the patient's face that is affected by a particular procedure, for example.

Registration is the process by which color and texture image content is first mapped to facial contour information, and further how the combined facial contour and color/texture content can then be mapped to the 3-D information that is provided by CBCT volume imaging. Various techniques for registration of image content from different sources are known to those skilled in the image processing arts and can be adapted to this problem for the imaging apparatus of the present disclosure.

With respect to the camera(s) used to capture reflectance image content, it is useful to have calibration information that relates to the optical geometry of image acquisition. This type of intrinsic information includes data describing parameters such as focal length, imaging length (depth of field), image center, field of view, and related metrics. An initial calibration of the camera can be performed to identify optical characteristics, such as using a set of targets and executing an imaging sequence that captures image data from representative angles, for example.

Additional, extrinsic information relates the position of the imaging subject at head supporting position 16 (FIG. 1) to real-world coordinates for each type of imaging system that is used. Extrinsic geometry includes positional information on spatial location, angle, and related metrics for camera, lens, filter, and other optical components, relative to the head supporting position 16. Extrinsic geometry can be obtained by reconstructing a coarse point cloud using a set of color images from representative angles, then registering the coarse point cloud to a 3-D dense mesh obtained from contour image processing.

It should be noted that a full orbit about the head of the patient is generally not needed for providing volume or contour information using apparatus and methods of the present disclosure. Imaging from one side of the head to the other may be sufficient for providing the needed depth information.

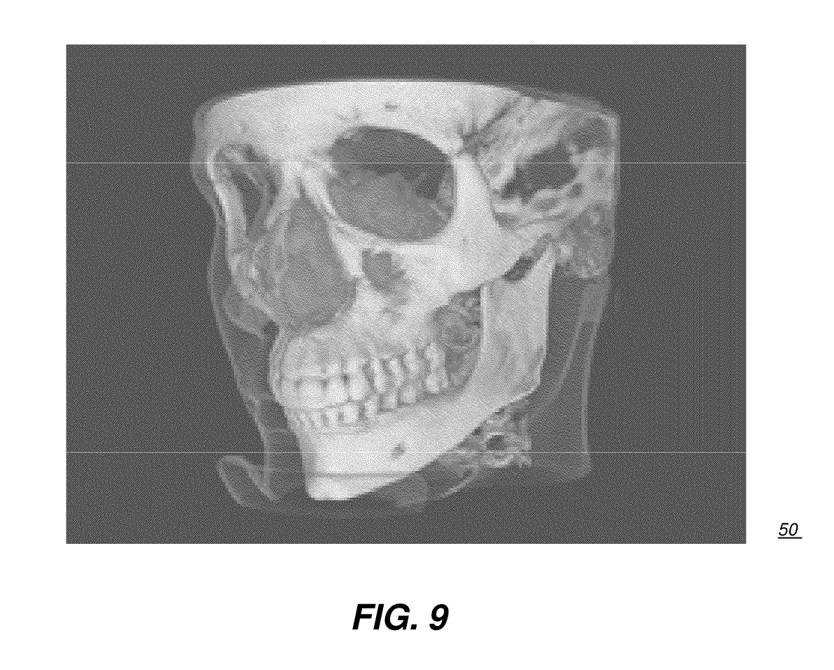

FIG. 9 shows a composite image 50 that includes both radiographic volume image content and contour and color reflectance image content. Display utilities on display 40 (FIG. 1) give the viewing operator the option to control transparence or density of the external surface content from reflectance images or of the volume image content from CBCT processing.

Consistent with one embodiment, the present invention utilizes a computer program with stored instructions that control system functions for image acquisition and image data processing for image data that is stored and accessed from an electronic memory. As can be appreciated by those skilled in the image processing arts, a computer program of an embodiment of the present invention can be utilized by a suitable, general-purpose computer system, such as a personal computer or workstation that acts as an image processor, when provided with a suitable software program so that the processor operates to acquire, process, and display data as described herein. Many other types of computer systems architectures can be used to execute the computer program of the present invention, including an arrangement of networked processors, for example.

The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present invention may also be stored on computer readable storage medium that is connected to the image processor by way of the internet or other network or communication medium. Those skilled in the image data processing arts will further readily recognize that the equivalent of such a computer program product may also be constructed in hardware.

It is noted that the term “memory”, equivalent to “computer-accessible memory” in the context of the present disclosure, can refer to any type of temporary or more enduring data storage workspace used for storing and operating upon image data and accessible to a computer system, including a database. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer can also be considered to be a memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It is understood that the computer program product of the present invention may make use of various image manipulation algorithms and processes that are well known. It will be further understood that the computer program product embodiment of the present invention may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present invention, are not specifically shown or described herein and may be selected from such algorithms, systems, hardware, components and elements known in the art.

Exemplary embodiments according to the application can include various features described herein (individually or in combination).

One apparatus embodiment for imaging the head of a patient, can include a transport apparatus that is configured to move an x-ray source and a detector about a head supporting position in at least partial orbit about the supported patient's head for obtaining a plurality of two-dimensional radiographic images at different angles relative to the head supporting position; a light source coupled to the transport apparatus and energizable to project a structured near infrared light pattern to a light conditioning element and toward the head supporting position over at least a portion of the orbit; a camera coupled to the transport apparatus and configured to record, at each of a plurality of angles of the orbit, a reflectance image of the structured light pattern that is projected against the supported patient's head; and a control logic processor that energizes at least the x-ray source, the x-ray detector, the transport apparatus, the light source, and the camera to acquire and process both radiographic and reflectance near infrared image data obtained during the at least partial orbit about the head supporting position, wherein the control logic processor is configured to process the obtained two-dimensional radiographic images for generating a volume reconstruction and to process the reflectance near infrared image content to register one or more contour images to the volume reconstruction. In one embodiment, the camera is further configured to acquire one or more color reflectance images of the supported patient's head.

While the invention has been illustrated with respect to one or more implementations, alterations and/or modifications can be made to the illustrated examples without departing from the spirit and scope of the appended claims. In addition, while a particular feature of the invention can have been disclosed with respect to one of several implementations, such feature can be combined with one or more other features of the other implementations as can be desired and advantageous for any given or particular function. The term “at least one of” is used to mean one or more of the listed items can be selected. The term “about” indicates that the value listed can be somewhat altered, as long as the alteration does not result in nonconformance of the process or structure to the illustrated embodiment. Other embodiments of the invention will be apparent to those skilled in the art from consideration of the specification and practice of the invention disclosed herein. It is intended that the specification and examples be considered as exemplary only, with a true scope and spirit of the invention being indicated by the following claims, and all changes that come within the meaning and range of equivalents thereof are intended to be embraced therein.

What is claimed is:

1. An apparatus for imaging the head of a patient, comprising:
a transport apparatus that moves an x-ray source and an x-ray detector in at least partial orbit about a head supporting position for the patient for acquiring, at each of a plurality of angles about the supporting position, a 2-D radiographic projection image of the patient's head;
a light source coupled to the transport apparatus and energizable to project a patterned light toward the head supporting position over at least a portion of the orbit;
a monochrome camera coupled to the transport apparatus and disposed to record, at each of one or more angles of the orbit, a monochrome reflectance image of the projected patterned light against the patient's head;
a color camera coupled to the transport apparatus and disposed to acquire, at each of one or more angles of the orbit, a color reflectance image of the patient's head at the head supporting position; and
a control logic processor that energizes at least the x-ray source, the detector, the transport apparatus, the light source, and the monochrome and color cameras to acquire and process both radiographic and reflectance image data obtained during the at least partial orbit about the head supporting position.

2. The apparatus of claim 1 further comprising a display in signal communication with the control logic processor for displaying combined radiographic and reflectance image content.

3. The apparatus of claim 1 wherein the light source is a solid-state light source.

4. The apparatus of claim 1 wherein the light source emits light in the near infrared region.

5. The apparatus of claim 1 further comprising one of a grating, a Powell lens, and a spatial light modulator in the path of light from the light source for forming the patterned light.

6. The apparatus of claim 1 further comprising an interference filter in the path of reflectance light to the color camera.

7. The apparatus of claim 1 wherein the x-ray source and detector provide cone beam computed tomography imaging.

8. The apparatus of claim 1 further comprising a polychromatic light source that is energizable for obtaining an image from the color camera.

9. An apparatus for imaging the head of a patient, comprising:
a transport apparatus that is configured to move an x-ray source and a detector about a head supporting position in at least partial orbit about the supported patient's head for obtaining two-dimensional radiographic images;
a laser light source coupled to the transport apparatus and energizable to project a monochrome near infrared light pattern toward the head supporting position over at least a portion of the orbit;
a monochrome camera coupled to the transport apparatus and configured to record, at one or more angles of the orbit, a monochrome reflectance image of the monochrome near infrared light pattern that is projected against the supported patient's head;
a color camera coupled to the transport apparatus, wherein the color camera is disposed to acquire, at each of one or more angles of the orbit, a color reflectance image of the supported patient's head; and
a control logic processor that energizes at least the x-ray source, the x-ray detector, the transport apparatus, the laser light source, and the monochrome and color cameras to acquire and process both radiographic and reflectance image data obtained during the at least partial orbit about the head supporting position, wherein the control logic processor is configured to process the obtained two-dimensional radiographic images for generating a volume reconstruction and to register contour and color information from the monochrome and color cameras to the volume reconstruction.

10. The apparatus of claim 9 further comprising a color display in signal communication with the control logic processor for displaying the volume reconstruction combined with reflectance image content.

11. The apparatus of claim 9 further comprising an interference filter in the path of reflectance light to the color camera.

12. The apparatus of claim 9 wherein the x-ray source and detector provide cone beam computed tomography imaging.

13. The apparatus of claim 9 wherein the monochrome near infrared light pattern is a two-dimensional pattern.

14. A method for imaging the head of a patient, the method executed at least in part by a computer and comprising:
orbiting an x-ray source and an x-ray detector in at least partial orbit about a head supporting position for the patient;
acquiring, at each of a plurality of angles about the supporting position, a 2-D radiographic projection image of the patient's head;
energizing a light source that orbits with the x-ray source and detector to project a light pattern toward the head supporting position over at least a portion of the orbit;
recording, at one or more angles of the orbit, a reflectance image of the projected light pattern against the patient's head;
acquiring at one or more angles of the orbit, a color reflectance image of the patient's head at the head supporting position;
reconstructing a volume image from the acquired 2-D radiographic projection images;
forming a contour image according to the recorded reflectance images of the light pattern;
registering the contour image to the reconstructed volume image;
mapping image content from one or more of the color reflectance images to the registered contour image; and
displaying a composite image that shows the mapped reflectance image content in registration with the corresponding volume image content.

15. The method of claim 14 wherein the light source emits near infrared light.

16. The method of claim 14 wherein the energized light source forms the light pattern using a spatial light modulator or a grating to form the structured light pattern.

17. The method of claim 14 wherein the energized light source is a solid-state light source.

18. The method of claim 14 wherein a single camera is used for recording both the reflectance image of the structured light pattern and the color reflectance images.

19. The method of claim 14 wherein the structured light pattern is formed by scanning a laser beam.

20. The method of claim 14 wherein the structured light pattern is projected as a two-dimensional pattern.
